(19) United States
(12) Patent Application Publication
(10) Pub. No.: US 2023/0048862 A1
(43) Pub. Date: Feb. 16, 2023

(54) STERILIZATION FABRIC PROCESSING EQUIPMENT AND PRODUCT MANUFACTURED BY THE SAME

(71) Applicant: MU-CHUN TSAI, NEW TAIPEI CITY (TW)

(72) Inventor: MU-CHUN TSAI, NEW TAIPEI CITY (TW)

(21) Appl. No.: 17/735,132

(22) Filed: May 3, 2022

(30) Foreign Application Priority Data
Aug. 16, 2021 (TW) 110209647

(51) Int. Cl. A61L 2/07; A61L 2/16; A61L 2/24

(52) U.S. Cl. CPC A61L 2/07; A61L 2/16; A61L 2/24; A61L 2202/26; A61L 2202/122; A61L 2202/14; A61L 2202/15

(57) ABSTRACT

Disclosed are a sterilization fabric processing equipment and a product manufactured by the processing equipment. The processing equipment includes a processing cavity, a steam device, a chemical tank, a pump set, and an automatic control device. The processing cavity has a working door and an interior for putting a frame and a base fabric overlaid on the frame. The steam device is for delivering steam to the processing cavity. The chemical tank is provided for containing a functional ion chemical and communicated with the steam pipeline through the chemical pipeline. The pump set is for delivering the steam and functional ions into the processing cavity. Under the effect of the predetermined pressure, the functional ions are closely combined to the heated base fabric fiber. The automatic control device is telecommunicatively connected to the steam device and the pump set for controlling their operation.

### CROSS-REFERENCE TO RELATED APPLICATIONS

This non-provisional application claims priority under 35 U.S.C. § 119(a) on Patent Application No(s). 110209647 filed in Taiwan, R.O.C. on Aug. 16, 2021, the entire contents of which are hereby incorporated by reference.

### BACKGROUND

### Technical Field

The present disclosure relates to the field of fabrics, and more particularly to a novel sterilization fabric processing equipment and a product manufactured by the equipment, and the equipment is capable of manufacturing a fabric with high durability, high washability, and long service life.

### Description of Related Art

With the evolution of technology and the improvement of life quality, people have increasingly higher requirements for the function of various products. For example, people want to expand or improve the functions of fabrics of textiles, in addition to the basic functions. In order to have more diverse functions of the fabrics, the fabrics are usually immersed, coated, or glued in order to attach particles or ions with special functions onto the surface of the fabrics. For example, the required particles or ions are diluted in a solvent to form a liquid first, and then the liquid is attached onto the surface of the fabrics by dipping or spraying. Although this method has the advantages of low cost and high manufacturing efficiency, the particles or ions cannot be firmly fixed onto the fabrics by these manufacturing methods. As a result, the particles or ions may fall off easily by external forces during use, or the particles or ions may be washed away during washing, which will cause the fabric to lose the desired functions in a short time and greatly reduce its service life.

In view of the aforementioned drawbacks, the present discloser based on years of experience in the related industry to conduct extensive research and experiment and finally developed a sterilization fabric processing equipment and a product manufactured by the equipment to provide a durable and high-quality fabric product.

### SUMMARY

Therefore, it is a primary objective of the present disclosure to provide a sterilization fabric processing equipment and a product manufactured by the equipment with a novel processing technology, so as to enhance the binding strength between the functional ions and the fabric fibers, and improve the quality, durability and service life of the product manufactured by the equipment.

To achieve the aforementioned and other objective, the present disclosure provides a sterilization fabric processing equipment for processing a base fabric which is overlaid on a frame, and the equipment includes: a processing cavity, having a working door, for putting the frame and the base fabric therein, and the working door being used for forming an airtight state inside the processing cavity when the working door is closed;

a steam device, connected to the processing cavity through a steam pipeline, for delivering steam into the processing cavity, and keeping the interior of the processing cavity at a predetermined pressure and a predetermined temperature by the steam; a chemical tank, provided for containing at least one functional ion chemical, and communicated with the steam pipeline through the chemical pipeline to deliver the functional ion chemical to the processing cavity; a pump set, communicated to the chemical pipeline and the steam pipeline, for sending steam and the functional ion chemical into the processing cavity, wherein after the functional ion chemical is sent into the processing cavity, the functional ions in the functional ion chemical under the effect of the predetermined pressure are combined tightly with fibers of the heated base fabric; and an automatic control device, telecommunicatively connected to the steam device and the pump set, for controlling of an operation state of the steam device and the pump set. In this way, the predetermined pressure and temperature inside the processing cavity can be kept by the delivery of steam, which is beneficial to heat up the base fabric, and the predetermined pressure promotes the combination of the functional ions and the fibers of the base fabric to greatly enhance the binding strength of the functional ions, so that the finished product has excellent washability and wear resistance, and the functional ions will not fall off easily even after repeated washing, and the effect of maintaining the fabric function for a long time can be achieved.

Preferably, the sterilization fabric processing equipment further includes a detection device set installed in the processing cavity for monitoring and controlling the pressure and temperature inside the processing cavity to facilitate controlling the internal state of the processing cavity at any time. Preferably, the detection device set is telecommunicatively connected to the automatic control device for reporting the internal pressure and temperature of the processing cavity to the automatic control device to allow the automatic control device to adjust the operating state of the steam device and the pump set at any time, so as to maintain the interior of the processing cavity at the environmental conditions of the predetermined pressure and temperature.

Preferably, the detection device set further includes a pressure gauge and a temperature gauge, for instantly displaying the values of the internal pressure and temperature of the processing cavity. In this way, an operator can know the pressure and temperature inside the processing cavity and adjust each processing parameter in real time.

Preferably, the processing cavity contains at least one guide structure provided for guiding the frame into the processing cavity along the guide structure in order to move the frame into the processing cavity smoothly and stably.

Preferably, there are two guide structures symmetrically disposed on a bottom side of the processing cavity, and each of the guide structures is a slide rail, which is suitable for most common movable frames.

Preferably, the automatic control device the automatic control device is telecommunicatively connected to the working door for automatically controlling an open state and a closed state of the working door in order to achieve a more comprehensive automatic control operation.

Preferably, the processing cavity further has a pressure relief valve telecommunicatively connected to the automatic control device and driven by the automatic control device for controlling an operation state of the pressure relief valve to improve the control precision of the predetermined pressure.

Preferably, the predetermined pressure is 2 Kg, the predetermined temperature is 130 degrees Celsius, and the processing time of the base fabric is 40 minutes, and these parameters can provide better conditions for tightly combining the functional ions with the base fabric fiber while preventing damages to the base fabric.

Preferably, the functional ion chemical is one selected from the silver ion, titanium ion, copper ion, or their combinations and has an excellent sterilization efficacy.

The present disclosure also provides a sterilization fabric manufactured by the aforementioned sterilization fabric processing equipment, and the sterilization fabric includes a base fabric, having a plurality of fibers; and a plurality of functional ions combined with the fibers respectively.

As described above, the fabric manufactured by the sterilization fabric processing equipment has excellent durability, and the functional ions on the fiber have a very strong binding strength. In other words, the sterilization fabric can resist multiple washings and prevent damages caused by external forces, and the functional ions can exist for a long time, thereby greatly improving the quality and service life of the sterilization fabric and overcoming the drawbacks of the present existing products.

In summation, the sterilization fabric processing equipment and the product manufactured by the equipment in accordance with the present disclosure use a closed environment of the processing cavity in conjunction with the delivery of time, such that the functional ions can be closely combined with the heated base fabric fiber in response to the predetermined pressure caused by the steam. Compared with the traditional immersion, coating or gluing method, the sterilization fabric manufactured by the processing equipment in accordance with this disclosure features excellent washability and wear resistance and can effectively prevent the traditional fabric from falling off due to repeated washing or external factors and prevent adverse affections to the function of the fabric. Further, the present disclosure also proposes many other technical features and advantages as described above.

### BRIEF DESCRIPTION OF THE DRAWINGS

FIG. 1 is a schematic view of a sterilization fabric processing equipment in accordance with a preferred embodiment of the present disclosure;

FIG. 2 is a schematic view showing an application of a sterilization fabric processing equipment in accordance with a preferred embodiment of the present disclosure;

FIG. 3 is a schematic view showing the structure of a sterilization fabric in accordance with a preferred embodiment of the present disclosure; and

FIG. 4 is a schematic view showing a partial structure of a sterilization fabric in accordance with a preferred embodiment of the present disclosure.

### DESCRIPTION OF THE EMBODIMENTS

This disclosure will now be described in more detail with reference to the accompanying drawings that show various embodiments of this disclosure.

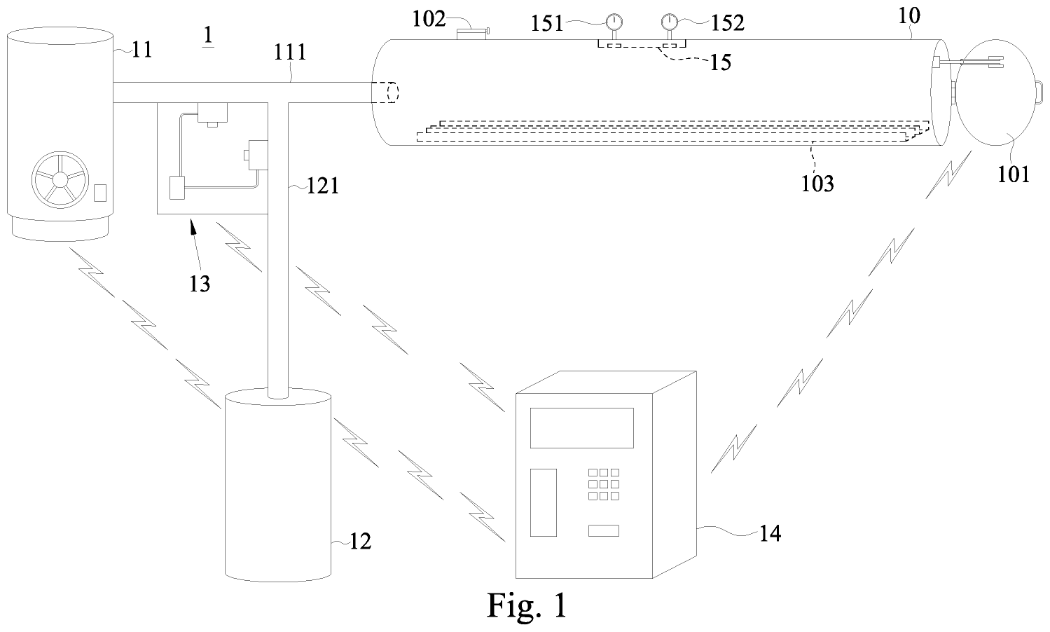

With reference to FIGS. 1, 2, 3 and 4 for the schematic views showing a sterilization fabric processing equipment, an application of the sterilization fabric processing equipment, a structure of a sterilization fabric and a partial structure of the sterilization fabric in accordance with the preferred embodiment of the present disclosure respectively, the dimensions, size and distance as shown in the figure are intended for the purpose of illustrating the technical characteristics of the present disclosure only, but not for the purpose of representing the actual structure. The present disclosure provides a sterilization fabric processing equipment 1 for processing a base fabric 2, wherein the base fabric 2 is overlaid on a frame 3. Preferably, the base fabric 2 is overlaid in an up-and-down staggered manner on the frame 3 to facilitate the processing process, such that the base fabric 2 is distributed in an S-shape when viewed from the side. The sterilization fabric processing equipment 1 includes a processing cavity 10, a steam device 11, a chemical tank 12, a pump set 13 and an automatic control device 14.

The processing cavity 10 has a working door 101, and the interior of the processing cavity 10 is provided for putting the frame 3 and the base fabric 2 overlaid on the frame 3, and the working door 101 is provided for forming an airtight state inside the processing cavity 10 when the working door 101 is closed. The steam device 11 is connected to the processing cavity 10 through a steam pipeline 111 for delivering steam into the processing cavity 10, such that a predetermined pressure and a predetermined temperature are maintained inside the processing cavity 10 due to the steam. The chemical tank 12 is provided for containing at least one functional ion chemical 4 and connected to the steam pipeline 111 through a chemical pipeline 121 for delivering the functional ion chemical 4 into the processing cavity 10. The pump set 13 is communicated with the chemical pipeline 121 and the steam pipeline 111 for sending the steam and the functional ion chemical 4 into the processing cavity 10. After the functional ion chemical 4 is sent into the processing cavity 10, the functional ions 40 in the functional ion chemical 4 under the predetermined pressure are closely combined with the fiber 20 of the heated base fabric 2. The automatic control device 14 is telecommunicatively connected to the steam device 11 and the pump set 13 for controlling the operation of the steam device 11 and the pump set 13. Here, the term “telecommunicatively connected” used throughout this specification refers to a connection that allows a control signal to be transmitted between devices via a cable or wireless transmission depending on actual needs.

After being processed by the sterilization fabric processing equipment 1, the functional ions 40 can be securely combined with the fibers 20 of the base fabric 2 to achieve the effects of improving the product quality and function retention time of the manufactured fabric product, greatly reducing the chance of ions falling off due to washing or external forces, featuring excellent washability of the sterilization fabric 9, extending the service life of the sterilization fabric 9, and providing a substantially permanent sterilization. During processing, the sterilization fabric processing equipment 1 uses the processing cavity 10 in conjunction with the steam to maintain the predetermined temperature and pressure in the closed processing cavity 10, and the base fabric 2 placed in the processing cavity 10 is heated by the steam to form a fiber opening state which is similar to the opening of pores of the fibers 20, and the functional ion chemical 4 in the chemical tank 12 is atomized due to the steam to distribute the functional ions in the processing cavity 10 and pressed into the fibers 20 by the predetermined pressure, so as to tightly combine the functional ions with the fibers 20. After the processing is completed, or the predetermined pressure and temperature inside the processing cavity 10 are reached, the automatic control device 14 will stop delivering the steam and the functional ion chemical 4. After the processing is completed and the fibers 20 of the base fabric 2 are cooled, the fibers 20 will be in a fiber closing state, so that the functional ions 40 and the fibers 20 are securely combined with each other to produce a powerful functional fabric.

In order to know the conditions in the processing cavity 10 more accurately, the sterilization fabric processing equipment 1 further includes a detection device set 15 installed in the processing cavity 10 for monitoring and controlling the pressure and temperature in the processing cavity 10 to control the status of the processing cavity 10 at any time, wherein the detection device set 15 can be implemented in the form of being telecommunicatively connected to a factory control center, an operator's electronic device or a warning device, so that an alarm can be issued when abnormality occurs. The detection device set 15 can be telecommunicatively connected to the automatic control device 14 in order to report the pressure and temperature inside the processing cavity 10 to the automatic control device 14, so that the automatic control device 14 can compare the actual conditions reported by the detection device set 15 according to the predetermined temperature and pressure required by the processing, and accurately adjust the operation status of the pump set 13 and the steam device 11 at any time, so as to surely maintain the required operation environment in the processing cavity 10.

In addition, the detection device set 15 further includes a temperature gauge 151 and a pressure gauge 152 for displaying the pressure and temperature values in real time and allowing factory operators to view and confirm the pressure and temperature directly.

In an embodiment, the automatic control device 14 is telecommunicatively connected to the working door 101 for automatically controlling the open and closed states of the working door 101. In this way, the overall processing flow can be simplified. After the base fabric 2 and the frame 3 are placed in the processing cavity 10, the working door 101 can be closed automatically by the automatic control device 14, and a closed space is formed inside the processing cavity 10. On the other hand, the working door 101 can be opened automatically after the processing is completed, without requiring a manual operation by the operator to provide a full automation for the whole process. Further, a sensing element is telecommunicatively connected to the automatic control device 14 and provided for automatically confirming whether the base fabric 2 and the frame 3 are in the processing positions, and after the base fabric 2 and the frame 3 are in the predetermined positions, the working door 101 can be controlled and closed automatically.

In another embodiment, the processing cavity 10 further has a pressure relief valve 102 telecommunicatively connected to the automatic control device 14 and controlled by the automatic control device 14 for its operation. The pressure relief valve 102 can be used to quickly release the pressure in the processing cavity 10. For example, the automatic control device 14 can drive the pressure relief valve 102 to reduce the pressure inside the processing cavity 10 when the set processing time is reached, and then the working door 101 is opened in order to take out the sterilization fabric 9.

Preferably, the processing cavity 10 contains at least one guide structure 103 for guiding the frame 3 along the guide structure 103 into the processing cavity 10, so as to facilitate moving the frame 3 and the base fabric 2 overlaid on the frame 3 quickly and stably, and positioning them at the predetermined positions. In this embodiment, there are two guide structures 103 arranged symmetrically on the bottom side of the processing cavity 10, and the guide structures 103 are slide rails, not only allowing the frame 3 to slide along the guide structures 103, but also achieving the effects of limiting position and preventing offset. Especially for the processing of a large-area base fabric 2, the frame 3 used to carry the base fabric 2 will also be very large. With the arrangement of the guide structures 103, the frame 3 can slide more stably and smoothly into the processing cavity 10, and avoid the situation of skew placement or difficult movement, etc., and the sliding rail-like structure can also limit the moving path of the frame 3. It is noteworthy that FIG. 1 is a schematic view of a preferred embodiment of the guide structures 103 only, but this figure is not intended for the purpose of showing the actual structure of the guide structures 103.

The sterilization fabric processing equipment 1 can carry out the required function addition operation for various kinds of the base fabric 2, and the functional ion chemical 4 in the chemical tank 12 can be silver ions, titanium ions, copper ions, zinc ions or any combination of the above. When several functional ion chemicals 4 are selected in the present disclosure, the functional ion chemicals 4 can be mixed in the chemical tank 12 and then pumped into the processing cavity 10 by the pump set 13 all at a time.

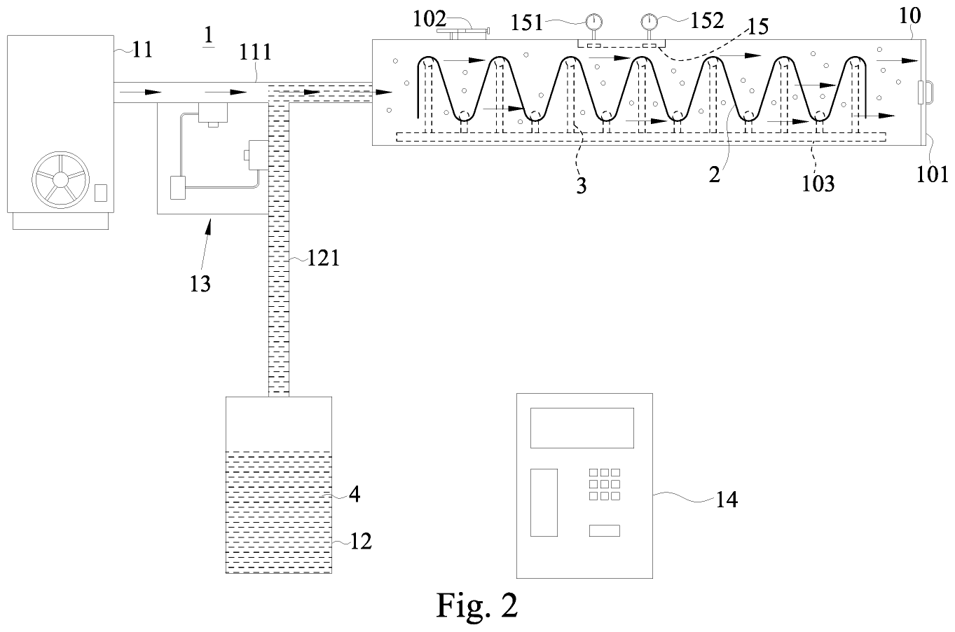

With reference to FIG. 2 for a preferred embodiment of the sterilization fabric processing equipment 1, the base fabric 2 set on the frame 3 is put into the processing cavity 10 first. When the processing cavity 10 has the guide structure 103, the guide structure 103 can be used as a positioning and moving aid for the frame 3 to allow the frame 3 to be placed at a predetermined position more easily, and then the working door 101 is closed to form an airtight state of the processing cavity 10. At this time, conditions such as the required processing time, temperature, pressure and chemical consumption can be set by the automatic control device 14; empirically, the preferred processing conditions include a predetermined pressure of 2 Kg, a predetermined temperature of 130 degrees Celsius, a processing time of 40 minutes and ensure that the functional ions 40 can be firmly combined with the fibers 20 of the base fabric 2, while preventing the base fabric 2 from being damaged. After setup, the automatic control device 14 will control the operation of the steam device 11 and pump set 13 according to the setting conditions, such that the predetermined temperature and pressure in the processing cavity 10 can be achieved, and the required amount of the functional ion chemical 4 can be delivered into the processing cavity 10. The detection device set 15 is operated continuously to detect the internal conditions of the processing cavity 10. After the predetermined temperature and pressure inside the processing cavity 10 and the required amount of the functional ion chemical 4 have reached the set values, the automatic control device 14 will stop delivering the steam and the functional ion chemical 4 and will sit still. The functional ions 40 in the functional ion chemical 4 will be pressed and tightly combined with the fibers 20 of the base fabric 2 due to the predetermined pressure. After the set processing time has reached, the pressure relief valve 102 will be opened to reduce the pressure in the processing cavity 10. The manufactured sterilization fabric 9 can be taken out after the working door 101 is opened.

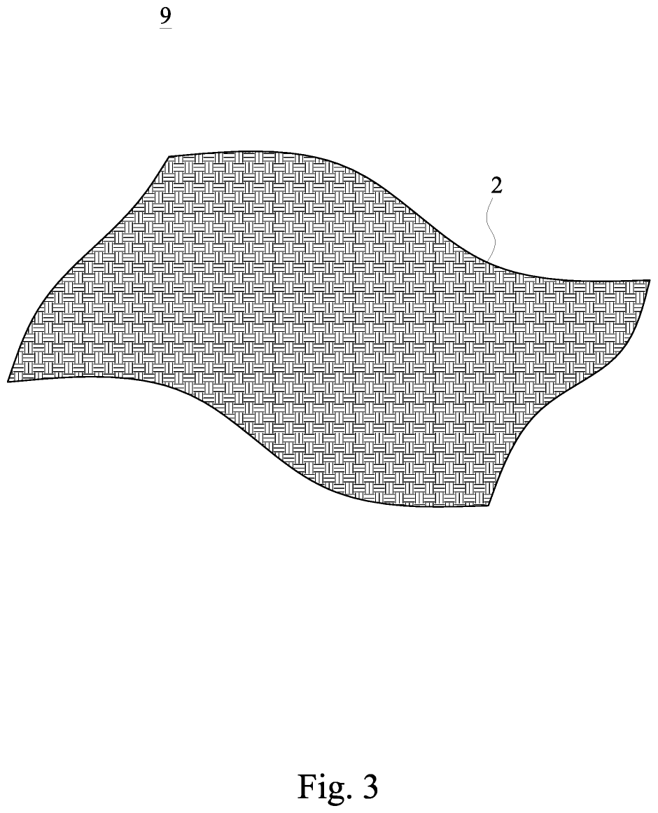

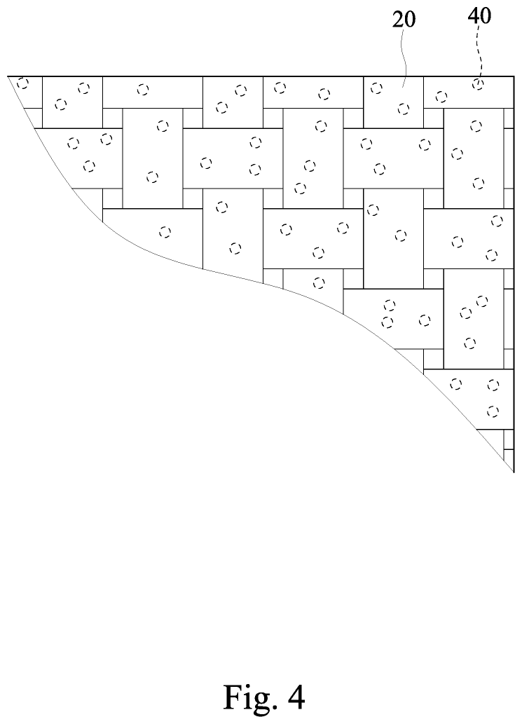

In FIGS. 3 and 4, the sterilization fabric 9 manufactured by the sterilization fabric processing equipment 1 specifically includes a base fabric 2 and a plurality of functional ions 40, and the base fabric 2 has a plurality of fibers 20, and the functional ions 40 are combined with the fibers 20 respectively. Since the predetermined pressure and temperature are formed in the sterilization fabric processing equipment 1 by the steam, so that the fibers 20 of the base fabric 2 can be heated to provide an effect similar to a fiber opening effect, and the functional ions 40 in the functional ion chemical 4 can be pressed into the fibers 20 under the effect of the predetermined pressure. Finally, the fibers 20 are cooled and folded again, so that the functional ions 40 can be fixed into the fibers 20 to produce a long-lasting sterilization fabric 9 which will not be affected by external forces easily.

In summation of the description above, the sterilization fabric processing equipment and product of the present disclosure uses the closed environment of the processing cavity together with the delivery of steam to create a predetermined pressure for the functional ions which are tightly combined with the heated base fabric fibers. Compared with the traditional immersion, coating and gluing solutions, the sterilization fabric manufactured by the processing equipment of the present disclosure has excellent washability and wear resistance, which can effectively prevent the chemical from falling off from to the fabric after repeated washings or by external factors, or affecting the function of the fabrics. In order to improve the control of the sterilization fabric processing equipment and the convenience and accuracy of its operation, the present disclosure further provides detailed technical characteristics such as the detection device set used to confirm the temperature and pressure at any time, or the guide structure used in the processing cavity to improve the convenience and stability of transferring the fabrics or the control of the working door and the internal pressure of the processing cavity and the optimal processing conditions, so as to improve the operational reliability of the sterilization fabric processing equipment and the product quality of the sterilization fabrics.

What is claimed is:

1. A sterilization fabric processing equipment, for processing a base fabric which is overlaid on a frame, comprising:
a processing cavity, having a working door, and provided for putting the frame and the base fabric therein, and the working door being used for forming an airtight state inside the processing cavity when the working door is closed;
a steam device, coupled to the processing cavity through a steam pipeline, for delivering steam into the processing cavity, and keeping interior of the processing cavity at a predetermined pressure and a predetermined temperature by the steam;
a chemical tank, provided for containing at least one functional ion chemical, communicated with the steam pipeline through the chemical pipeline to deliver the functional ion chemical to the processing cavity;
a pump set, communicated to the chemical pipeline and the steam pipeline, for sending steam and the functional ion chemical into the processing cavity, wherein after the functional ion chemical is sent into the processing cavity, functional ions in the functional ion chemical are combined tightly with heated fibers of the base fabric under the effect of the predetermined pressure; and
an automatic control device, telecommunicatively coupled to the steam device and the pump set, for controlling an operation state of the steam device and the pump set.

2. The sterilization fabric processing equipment according to claim 1, further comprising a detection device set, installed in the processing cavity, for monitoring and controlling the pressure and temperature inside the processing cavity.

3. The sterilization fabric processing equipment according to claim 2, wherein the detection device set further comprises a pressure gauge and a temperature gauge, for instantly displaying the values of internal pressure and temperature of the processing cavity.

4. The sterilization fabric processing equipment according to claim 3, wherein the detection device set is telecommunicatively coupled to the automatic control device, for reporting the internal pressure and temperature of the processing cavity to the automatic control device.

5. The sterilization fabric processing equipment according to claim 4, wherein the processing cavity contains at least one guide structure, for guiding the frame along the guide structure into the processing cavity.

6. The sterilization fabric processing equipment according to claim 5, wherein the guide structure comes with a quantity of two, and the two guide structures are symmetrically arranged on a bottom side of the processing cavity, and each of the guide structures is a slide rail.

7. The sterilization fabric processing equipment according to claim 6, wherein the automatic control device is telecommunicatively coupled to the working door for automatically controlling an open state and a closed state of the working door.

8. The sterilization fabric processing equipment according to claim 7, wherein the processing cavity further has a pressure relief valve telecommunicatively coupled to the automatic control device, and driven by the automatic control device for controlling an operation state of the pressure relief valve.

9. The sterilization fabric processing equipment according to claim 8, wherein the predetermined pressure is 2 Kg, the predetermined temperature is 130 degrees Celsius, and the processing time of the base fabric is 40 minutes.

10. The sterilization fabric processing equipment according to claim 9, wherein the functional ion chemical is one selected from the group consisting of silver ion, titanium ion, copper ion, and zinc ion chemicals, or a combination thereof.

11. A sterilization fabric, manufactured by the sterilization fabric processing equipment according to claim 1, comprising:
a base fabric, having a plurality of fibers; and
a plurality of functional ions, combined with the fibers respectively.

12. The sterilization fabric according to claim 11, further comprising a detection device set, installed in the processing cavity, for monitoring and controlling the pressure and temperature inside the processing cavity.

13. The sterilization fabric processing equipment according to claim 12, wherein the detection device set further comprises a pressure gauge and a temperature gauge, for instantly displaying the values of internal pressure and temperature of the processing cavity.

14. The sterilization fabric according to claim 13, wherein the detection device set is telecommunicatively coupled to the automatic control device, for reporting the internal pressure and temperature of the processing cavity to the automatic control device.

15. The sterilization fabric according to claim 14, wherein the processing cavity contains at least one guide structure, for guiding the frame along the guide structure into the processing cavity.

16. The sterilization fabric according to claim 15, wherein the guide structure comes with a quantity of two, and the two guide structures are symmetrically arranged on a bottom side of the processing cavity, and each of the guide structures is a slide rail.

17. The sterilization fabric according to claim 16, wherein the automatic control device is telecommunicatively coupled to the working door for automatically controlling an open state and a closed state of the working door.

18. The sterilization fabric according to claim 17, wherein the processing cavity further has a pressure relief valve telecommunicatively coupled to the automatic control device, and driven by the automatic control device for controlling an operation state of the pressure relief valve.

19. The sterilization fabric according to claim 18, wherein the predetermined pressure is 2 Kg, the predetermined temperature is 130 degrees Celsius, and the processing time of the base fabric is 40 minutes.

20. The sterilization fabric according to claim 19, wherein the functional ions are selected from the group consisting of silver ions, titanium ions, copper ions, and zinc ions, or a combination thereof.
